(19) United States
(12) Patent Application Publication
(10) Pub. No.: US 2004/0024071 A1
(43) Pub. Date: Feb. 5, 2004

(54) Perovskite compositions and method of making and process of using such compositions

(76) Inventor: Paul F. Meier, Bartlesville, OK (US)

Correspondence Address: RICHMOND, HITCHCOCK, FISH & DOLLAR, P.O. Box 2443, Bartlesville, OK, 74005, US

(21) Appl. No.: 10/209,749

(22) Filed: Aug. 1, 2002

(51) Int. Cl. C07C 27/06; B01J 23/00

(52) U.S. Cl. 518/717; 502/325

(57) ABSTRACT

A novel composition containing a perovskite of the formula AB₁₋ₓB¹ₓ O₃ and having a Group VIII metal incorporated thereon is disclosed; wherein A is a Group IIIB metal, B is a Group VIII metal, and B¹ is a metal selected from the group consisting of the Group IVB metals, the Group VB metals, and the Group VIB metals.

The invention relates to compositions useful in synthesis gas conversion processes and to methods for their production and use.

### BACKGROUND OF THE INVENTION

Perovskites are mixed-metal oxides with wide latitude for substitution having a general formula of ABO₃. The use of perovskites in the conversion of synthesis gas to hydrocarbons such as methane, methanol and olefins is well known in the art. However, there are ever present incentives for the development of new, more effective and/or more practical perovskite compositions for use in converting synthesis gas, and methods of preparing them.

### SUMMARY OF THE INVENTION

It is an object of this invention to provide a perovskite containing composition effective for converting synthesis gas to hydrocarbons.

It is a further object of this invention to provide a method of preparing a perovskite containing composition effective for converting synthesis gas to hydrocarbons.

It is another object of this invention to employ this perovskite containing composition as a catalyst in the conversion of synthesis gas to hydrocarbons.

Yet another object of this invention is to increase the efficiency of the conversion of synthesis gas to hydrocarbons, and in particular, to olefinic hydrocarbons.

According to a first embodiment of the present invention, a perovskite containing composition which can be used in the conversion of synthesis gas is provided and comprises a first component comprising a Group VIII metal; and

a second component comprising:

a) a first metal selected from the group consisting of the Group III B metals;

b) a second metal selected from the Group VIII metals;

c) a third metal selected from the group consisting of the Group IVB metals, the Group VB metals, the Group VIB metals; and

d) oxygen.

According to a second embodiment of the present invention, a method which can be used for producing a perovskite containing composition is provided and comprises the steps of:

a) mixing:

(1) a first compound comprising a Group IIIB metal,

(2) a second compound comprising a Group VIII metal compound, and

(3) a third compound comprising a metal selected from the group consisting of a Group IVB metal, a Group VB metal and

a Group VIB metal, to thereby form a mixture thereof;

b) calcining said mixture to thereby form a calcined material; and

c) incorporating a Group VIII metal onto said calcined material to thereby form said composition.

According to a third embodiment of the present invention, a process for the conversion of synthesis gas is provided and comprises contacting synthesis gas with a composition in accordance with the first embodiment or the second embodiment, under reaction conditions, to thereby convert at least a portion of the synthesis gas to a hydrocarbon.

Other objects and advantages will become apparent from the detailed description and the appended claims.

### DETAILED DESCRIPTION OF THE INVENTION

According to the first embodiment of the present invention, the composition can comprise, consist essentially of, or consist of a first component comprising a Group VIII metal; and a second component comprising: a) a first metal selected from the group consisting of the Group III B metals; b) a second metal selected from the Group VIII metals; c) a third metal selected from the group consisting of the Group IVB metals, the Group VB metals, and the Group VIB metals; and d) oxygen. The reference to Group metals herein refers to the CAS version of the Periodic Table of the Elements.

The composition preferably has the formula AB₁₋ₓB¹ₓO₃ wherein A is said first metal, B is said second metal, and B¹ is said third metal. Preferably, x is greater than about 0.2, and x is less than about 0.7; more preferably x is greater than about 0.3 and x is less than about 0.6; even more preferably x is greater than about 0.4 and x is less than about 0.6; and most preferably x equals 0.5.

The Group VIII metal of the first component preferably comprises a metal selected from the group consisting of iron, cobalt, ruthenium, nickel, and combinations of any two or more thereof, and most preferably comprises iron.

The first metal of the second component preferably comprises lanthanum, and the second metal of the second component preferably comprises iron.

The third metal of the second component is preferably selected from the group consisting of titanium, vanadium, chromium, zirconium, molybdenum, and tungsten; and most preferably is selected from the group consisting of titanium, vanadium, chromium and zirconium.

The first component is preferably present in the composition in an amount in the range of from about 10 to about 30 weight %, more preferably in the range of from about 15 to about 25 weight %, and most preferably in the range of from about 18 to about 22 weight %, based on the total weight of the composition.

According to a second embodiment of the present invention, the inventive composition can be prepared by the following method comprising, consisting of, or consisting essentially of the following steps.

The first step comprises mixing:

(1) a first compound comprising a Group IIIB metal,

(2) a second compound comprising a Group VIII metal, and

(3) a third compound comprising a metal selected from the group consisting of a Group IVB metal, a Group VB metal and

a Group VIB metal, to thereby form a mixture thereof.

The mixing is preferably performed by physically mixing and grinding the compounds.

The mixture is then calcined to thereby form a calcined material. The calcining generally includes a temperature in the range of from about 800° C. to about 1000° C.; preferably from about 850° C. to about 950° C.; and most preferably about 900° C. The calcining is also preferably performed in the presence of air. The calcined material is then reground and calcined again, as described above.

The Group IIIB metal of the first compound preferably comprises lanthanum and the Group VIII metal of the second compound preferably comprises iron.

The metal of the third compound is preferably selected from the group consisting of titanium, vanadium, chromium, zirconium, molybdenum, and tungsten; and more preferably is selected from the group consisting of titanium, vanadium, chromium and zirconium.

A Group VIII metal is then incorporated onto the calcined material, to thereby form the composition, in any suitable manner known to those skilled in the art. The preferred method is by impregnation using any standard incipient wetness technique. Preferably, the Group VIII metal compound is dissolved in a liquid selected from water and a hydrocarbon. The Group VIII metal/liquid solution is then used to impregnate the calcined material by immersing the calcined material in the liquid solution for a time period suitable to incorporate the Group VIII metal onto the calcined material.

The Group VIII metal incorporated onto the calcined material preferably comprises a metal selected from the group consisting of iron, cobalt, ruthenium, nickel, and combinations of any two or more thereof; and more preferably comprises iron.

According to the third embodiment of the present invention, a process useful for converting synthesis gas to a hydrocarbon comprises, consists of, or consists essentially of contacting synthesis gas comprising, consisting of, or consisting essentially of carbon monoxide and hydrogen, with the inventive composition of either the first embodiment or the second embodiment, under reaction conditions, to thereby convert at least a portion of the synthesis gas to a hydrocarbon. Prior to use in the conversion of synthesis gas to hydrocarbons, the composition is preferably reduced using a reducing gas such as, but not limited to, hydrogen.

The reaction conditions include a temperature in the range of from about 260° C. to about 380° C.; preferably from about 280° C. to about 360° C.; and most preferably from about 300° C. to about 340° C.

Around fifty percent of the hydrocarbon produced generally contains in the range of from 2 to 4 carbon atoms per molecule.

### EXAMPLES

The following examples are intended to be illustrative of the present invention and to teach one of ordinary skill in the art to make and use the invention. These examples are not intended to limit the invention in any way.

### Example I

This example illustrates the preparation of catalysts that were subsequently tested in the conversion of synthesis gas to hydrocarbons.

Catalyst A (Control)

A 65 gram sample of solid lanthanum nitrate hexahydrate was ground and placed in a muffle furnace at 900° C. After 16 hours, the material was removed and ground again before reheating at 900° C. for another 16 hours. After this preparation procedure, X-ray Diffraction (XRD) showed that the material consisted essentially of La₂O₃. To prepare this material with approximately 20 wt % of iron, a 14.47 gram sample of Fe(NO₃)₃.9H₂O was added to deionized water and dissolved with heating and stirring. The solution was added by incipient wetness impregnation to 8 grams of the La₂O₃ using three separate additions. In between each addition, the impregnated catalyst was dried overnight at 110° C. under 0.5 atmosphere.

Catalyst B (Control)

A 65 gram sample of La(NO₃)₃.6H₂O and a 48.4 gram sample of Fe(NO₃)₃.9H₂O were physically mixed and ground. The material was then placed in a muffle furnace at 900° C. After 24 hours, the solid mixture was removed, ground, and mixed again before reheating at 900° C. for another 24 hours. This process was repeated over four days. After this preparation procedure, XRD showed that the material consisted of the LaFeO₃ perovskite with minor amounts of La₂O₃. To prepare this material with approximately 20 wt % of iron, a 14.47 gram sample of Fe(NO₃)₃9H₂O was added to deionized water and dissolved with heating and stirring. The solution was added by incipient wetness impregnation to 8 grams of the LaFeO₃ perovskite using three separate additions. In between each addition, the impregnated catalyst was dried overnight at 110° C. under 0.5 atmosphere.

Catalyst C (Invention)

A 65 gram sample of La(NO₃)₃.6H₂O, a 30.3 gram sample of Fe(NO₃)₃.9H₂O, and a 5.992 gram sample of TiO₂ were physically mixed and ground. The material was then placed in a muffle furnace at 900° C. After 24 hours, the solid mixture was removed, ground, and mixed again before reheating at 900° C. for another 24 hours. This process was repeated over four days. After this preparation procedure, XRD showed that the material consisted of the LaFe_{0 5}Ti_{0.5}O₃ perovskite with minor amounts of La(OH)₃ and La₂TiO₅. To prepare this material with approximately 20 wt % of iron, a 14.47 gram sample of Fe(NO₃)₃.9H₂O was added to deionized water and dissolved with heating and stirring. The solution was added by incipient wetness impregnation to 8 grams of the LaFe_{0.5}Ti_{0.5}O₃ perovskite using three separate additions. In between each addition, the impregnated catalyst was dried overnight at 110° C. under 0.5 atmosphere.

Catalyst D (Invention)

A 65 gram sample of La(NO₃)₃.6H₂O, a 30.3 gram sample of Fe(NO₃)₃.9H₂O, and a 6.821 gram sample of V₂O₅ were physically mixed and ground. The material was then placed in a muffle furnace at 900° C. After 24 hours, the solid mixture was removed, ground, and mixed again before reheating at 900° C. for another 24 hours. This process was repeated over four days. After this preparation procedure, XRD showed that the material consisted of the LaFe_{0.5}V_{0.5}O₃ perovskite with minor amount of LaVO₄. To prepare this material with approximately 20 wt % of iron, a 14.47 gram sample of Fe(NO₃)₃.9H₂O was added to deionized water and dissolved with heating and stirring. The solution was added by incipient wetness impregnation to 8 grams of the LaFe_{0.5}V_{0.5}O₃ perovskite using three separate additions. In between each addition, the impregnated catalyst was dried overnight at 110° C. under 0.5 atmosphere.

Catalyst E (Invention)

A 65 gram sample of La(NO₃)₃.6H₂O, a 30.3 gram sample of Fe(NO₃)₃.9H₂O, and a 18.537 gram sample of chromium acetate (Cr(C₂H₃O₂)₃.H₂O) were physically mixed and ground. The material was then placed in a muffle furnace at 900° C. After 24 hours, the solid mixture was removed, ground, and mixed again before reheating at 900° C. for another 24 hours. This process was repeated over four days. After this preparation procedure, XRD showed that the material consisted of the LaFe_{0.5}Cr_{0.5}O₃ perovskite. To prepare this material with approximately 20 wt % of iron, a 14.47 gram sample of Fe(NO₃)₃.9H₂O was added to deionized water and dissolved with heating and stirring. The solution was added by incipient wetness impregnation to 8 grams of the LaFe_{0.5}Cr0.5O₃ perovskite using three separate additions. In between each addition, the impregnated catalyst was dried overnight at 110° C. under 0.5 atmosphere.

Catalyst F (Control)

A 65 gram sample of La(NO₃)₃.6H₂O, a 30.3 gram sample of Fe(NO₃)₃.9H₂O, and a 18.38 gram sample of manganese acetate (Mn(C₂H₃O₂)₂.4H₂O) were physically mixed and ground. The material was then placed in a muffle furnace at 900° C. After 24 hours, the solid mixture was removed, ground, and mixed again before reheating at 900° C. for another 24 hours. This process was repeated over four days. After this preparation procedure, XRD showed that the material consisted of the LaFe_{0.5}Mn_{0.5}O₃ perovskite. To prepare this material with approximately 20 wt % of iron, a 14.47 gram sample of Fe(NO₃)₃.9H₂O was added to deionized water and dissolved with heating and stirring. The solution was added by incipient wetness impregnation to 8 grams of the LaFe_{0.5}Mn_{0.5}O₃ perovskite using three separate additions. In between each addition, the impregnated catalyst was dried overnight at 110° C. under 0.5 atmosphere.

Catalyst G (Invention)

A 65 gram sample of La(NO₃)₃.6H₂O, a 30.3 gram sample of Fe(NO₃)₃.9H₂O, and a 9.242 gram sample of ZrO₂ were physically mixed and ground. The material was then placed in a muffle furnace at 900° C. After 24 hours, the solid mixture was removed, ground, and mixed again before reheating at 900° C. for another 24 hours. This process was repeated over four days. After this preparation procedure, XRD showed that the material consisted of the LaFe_{0.5}Zr_{0.5}O₃ perovskite with minor amounts of La(OH)₃. To prepare this material with approximately 20 wt % of iron, a 14.47 gram sample of Fe(NO₃)₃.9H₂O was added to deionized water and dissolved with heating and stirring. The solution was added by incipient wetness impregnation to 8 grams of the LaFe_{0.5}Zr_{0.5}O₃ perovskite using three separate additions. In between each addition, the impregnated catalyst was dried overnight at 110° C. under 0.5 atmosphere.

### Example II

This example illustrates the use of the materials described in Example I as catalysts in the conversion of synthesis gas to hydrocarbons. In all cases, the following general procedure was used.

About 4 grams of the catalyst was sieved to 12-20 mesh and placed in a stainless steel tube reactor (length: about 17 inches; inner diameter: about 0.6 inches). From bottom to top, the reactor was first packed with a quartz wool plug, then alumina pellets, then quartz wool followed by the catalyst, quartz wool, and more alumina pellets for the preheat zone. The catalyst was centered in a 3-zone Thermcraft furnace. The temperature was controlled with a Eurotherm Model 91e, and monitored by a chromel alumel thermocouple positioned in the middle of the catalyst bed. Pressure was controlled with a Tescom 26-1700 and monitored with Pacific Scientific pressure gauges.

Catalyst activation was performed in the reactor with the first step being calcination at 450° C. with 60 cc/min air for 8 hours. The catalyst was then purged for 10 minutes with 60 cc/min nitrogen. Hydrogen was slowly added over a 10 minute period at a final flow rate of 60 cc/min while reducing the flow rate of nitrogen to zero. The temperature was then increased to 500° C. and the hydrogen reduction proceeded for 16 hours.

After completion of the catalyst activation procedure, the temperature of the catalyst was decreased to the specific reaction temperature being used. Matheson carbon monoxide (99.99% purity) was used without further purification. Hydrogen was purified with a Matheson 64-1008 series oxygen removal purifier followed by a Matheson 452 cartridge for removal of water. Gaseous flow rates were controlled and monitored with a Brooks mass flow controller, Monitor 0154E. The mass flow controllers for hydrogen and carbon monoxide were Brooks 5850 series. The catalyst was pressurized to 300 psig with a H₂/CO ratio of 1 and flow rates of 20 cc/min each. (This resulted in space velocity of about 480 GHSV).

A 10 cc receiver was used to collect product. The receiver was cooled to 0° C. in a wet ice bath that was constructed of 0.5 inch swagelok tubing and equipped with needle and shut-off valves. After completion of an experiment (usually 6 hour run), the valves were closed; the receiver was weighed and then slowly vented to collect liquid samples for gas chromatographic analysis. Gaseous product flow was measured every half-hour with a J&W Scientific ADM 2000 Flowmeter and the readings were averaged over the entire experiment. Gas phase products were analyzed using a Carle Series 400 AGC Model 196A GC while liquid samples were analyzed using an HP 6890 GC.

In Run I, 4.0 grams of Control catalyst A (20 wt % Fe on La₂O₃) described in Example I was tested at 320° C. and 300 psig using the procedure described above. The total feed passed over the catalyst was 8.2 grams. Results of this test are summarized in Table I.

In Run II, 4.0 grams of Control catalyst B (20 wt % Fe on LaFeO₃) described in Example I was tested at 320° C. and 300 psig using the procedure described above. The total feed passed over the catalyst was 8.9 grams. Results of these tests are summarized in Table I.

In Run III, 4.0 grams of Inventive catalyst C (20 wt % Fe on LaFe_{0.5}Ti_{0.5}O₃) described in Example I was tested at 320° C. and 300 psig using the procedure described above. The total feed passed over the catalyst was 6.5 grams. Results of this test are summarized in Table I.

In Run IV, 4.0 grams of Inventive catalyst D (20 wt % Fe on LaFe_{0.5}V_{0.5}O₃) described in Example I was tested at 320° C. and 300 psig using the procedure described above. The total feed passed over the catalyst was 6.5 grams. Results of this test are summarized in Table I.

Run V is an average of two separate experiments in which 4.0 grams of Inventive catalyst E (20 wt % Fe on LaFe_{0.5}Cr_{0.5}O₃) described in Example I was tested at 320° C. and 300 psig using the procedure described above. The average total feed passed over the catalyst was 7.1 grams. The average results of these tests are summarized in Table I.

In Run VI, 4.0 grams of Control catalyst F (20 wt % Fe on LaFe_{0.5}Mn_{0.5}O₃) described in Example I was tested at 320° C. and 300 psig using the procedure described above. The total feed passed over the catalyst was 8.9 grams. Results of this test are summarized in Table I.

In Run VII, 4.0 grams of Inventive catalyst G (20 wt % Fe on LaFe_{0.5}Zr_{0.5}O₃) described in Example I was tested at 320° C. and 300 psig using the procedure described above. The total feed passed over the catalyst was 7.1 grams. Results of this test are summarized in Table I.

TABLE I
Summary of Tests on Catalysts With 20 wt % Iron Added (320° C.)

| | Run | Run | Run | Run | Run | Run | Run |
|---|---|---|---|---|---|---|---|
| | I | II | III | IV | V | VI | VII |
| | La₂O₃ | LaFeO₃ | LaFe_{0 5}Ti_{0 5}O₃ | LaFe_{0 5}V_{0 5}O₃ | LaFe_{0 5}Cr_{0 5}O₃ | LaFe_{0 5}Mn_{0 5}O₃ | LaFe_{0 5}Zr_{0 5}O₃ |
| Catalyst | A | B | C | D | E | F | G |
| CO Wt % | | | | | | | |
| Conversion | 24.0 | 25.9 | 85.0 | 87.2 | 85.0 | 19.7 | 50.7 |
| CH₄ Wt % | | | | | | | |
| Selectivity | 7.3 | 8.8 | 22.9 | 17.8 | 18.5 | 1.5 | 10.7 |
| C₂-C₄ Wt % | 42.5 | 49.7 | 48.5 | 50.4 | 41.5 | 23.3 | 45.4 |
| Selectivity | | | | | | | |
| C5 + Wt % | | | | | | | |
| Selectivity | 37.4 | 31.0 | 23.4 | 28.1 | 33.1 | 30.1 | 17.0 |
| OH Wt % | | | | | | | |
| Selectivity | 12.8 | 10.4 | 5.2 | 3.7 | 6.9 | 45.1 | 26.8 |

In Run VIII, 4.0 grams of Control catalyst B (20 wt % Fe on LaFeO₃) described in Example I was tested at 340° C. and 300 psig using the procedure described above. The total feed passed over the catalyst was 8.2 grams. Results of these tests are summarized in Table II.

In Run IX, 4.0 grams of Inventive catalyst C (20 wt % Fe on LaFe_{0.5}Ti_{0.5}O₃) described in Example I was tested at 340° C. and 300 psig using the procedure described above. The total feed passed over the catalyst was 7.1 grams. Results of this test are summarized in Table II.

In Run X, 4.0 grams of Inventive catalyst D (20 wt % Fe on LaFe_{0.5}V_{0.5}O₃) described in Example I was tested at 340° C. and 300 psig using the procedure described above. The total feed passed over the catalyst was 7.1 grams. Results of this test are summarized in Table II.

In Run XI, 4.0 grams of Inventive catalyst E (20 wt % Fe on LaFe_{0.5}Cr_{0.5}O₃) described in Example I was tested at 340° C. and 300 psig using the procedure described above. The total feed passed over the catalyst was 8.5 grams. Results of this run are summarized in Table II.

Run XII is an average of two separate tests in which 4.0 grams of Inventive catalyst G (20 wt % Fe on LaFe_{0.5}Zr_{0.5}O₃) described in Example I was tested at 340° C. and 300 psig using the procedure described above. The total average feed passed over the catalyst was 7.1 grams. The average results of these tests are summarized in Table II.

TABLE II
Summary of Tests on Catalysts With 20 Wt % Iron Added (340° C.)

| | Run | Run | Run | Run | Run |
|---|---|---|---|---|---|
| | VIII | IX | X | XI | XII |
| | LaFeO₃ | LaFe_{0 5}Ti_{0.5}O₃ | LaFe_{0.5}V_{0.5}O₃ | LaFe_{0.5}Cr_{0.5}O₃ | LaFe_{0.5}Zr_{0.5}O₃ |
| Catalyst | B | C | D | B | E |
| CO Wt % | | | | | |
| Conversion | 63.9 | 83.8 | 84.6 | 94.2 | 90.4 |
| CH₄ Wt % | | | | | |
| Selectivity | 22.8 | 32.4 | 27.3 | 17.1 | 29.0 |
| C₂-C₄ Wt % | 55.8 | 51.3 | 44.6 | 25.6 | 54.0 |
| Selectivity | | | | | |
| C5 + Wt % | | | | | |
| Selectivity | 14.5 | 12.6 | 24.9 | 23.9 | 8.4 |
| OH Wt % | | | | | |
| Selectivity | 6.9 | 3.8 | 3.1 | 33.4 | 8.5 |

The test data presented in Table I show that the use of inventive catalysts C, D, E, and G in Runs III, IV, V, and VII, respectively, resulted in an increased activity as compared to the control catalysts A, B, and F in Runs I, II, and VI, respectively. For example, the three control catalysts (A, B, and F) in Control Runs I, II, and VI gave an average carbon monoxide conversion of 23.2 wt %. Inventive Runs III, IV, V, and VII demonstrated a 266 percent, 276 percent, 266 percent, and 119 percent increase in carbon monoxide conversion, respectively.

The test data presented in Table II show that even at the higher temperature of 340° C., the use of inventive catalysts C, D, E, and G in Runs IX, X, XI, and XII, respectively, resulted in an increased activity as compared to the control catalyst B in Run VIII (Table II). Inventive Runs IX, X, XI, and XII demonstrated a 31 percent, 32 percent, 47 percent, and 41 percent increase in carbon monoxide conversion, respectively, compared to Control Run VIII.

Reasonable variations, modification, and adaptations can be made within the scope of the disclosure and the appended claims without departing from the scope of this invention.

That which is claimed is:

1. A composition comprising:
a first component comprising a Group VIII metal; and
a second component comprising:
a) a first metal selected from the group consisting of the Group III B metals;
b) a second metal selected from the Group VIII metals;
c) a third metal selected from the group consisting of the Group IVB metals, the Group VB metals, and the Group VIB metals; and
d) oxygen.

2. A composition as recited in claim 1 wherein said Group VIII metal of said first component comprises a metal selected from the group consisting of iron, cobalt, ruthenium, nickel, and combinations of any two or more thereof.

3. A composition as recited in claim 1 wherein said Group VIII metal of said first component comprises iron.

4. A composition as recited in claim 1 wherein said first metal of said second component comprises lanthanum.

5. A composition as recited in claim 1 wherein said second metal of said second component comprises iron.

6. A composition as recited in claim 1 wherein said third metal of said second component is selected from the group consisting of titanium, vanadium, chromium, zirconium, molybdenum, and tungsten.

7. A composition as recited in claim 1 wherein said third metal of said second component is selected from the group consisting of titanium, vanadium, chromium and zirconium.

8. A composition as recited in claim 1 wherein said first component is present in said composition in an amount in the range of from about 10 to about 30 weight %, based on the total weight of said composition.

9. A composition as recited in claim 1 wherein said first component is present in said composition in an amount in the range of from about 15 to about 25 weight %, based on the total weight of said composition.

10. A composition as recited in claim 1 wherein said first component is present in said composition in an amount in the range of from about 18 to about 22 weight %, based on the total weight of said composition.

11. A composition as recited in claim 1 wherein said second component has the formula AB₁₋ₓB¹ₓO₃, wherein A is said first metal, B is said second metal, B¹ is said third metal, wherein x is greater than about 0.2, and wherein x is less than about 0.7.

12. A composition as recited in claim 11 wherein x is greater than about 0.3, and wherein x is less than about 0.6.

13. A composition as recited in claim 11 wherein x is greater than about 0.4, and wherein x is less than about 0.6.

14. A composition as recited in claim 11 wherein said Group VIII metal of said first component comprises a metal selected from the group consisting of iron, cobalt, ruthenium, and nickel.

15. A composition as recited in claim 11 wherein said Group VIII metal of said first component comprises iron.

16. A composition as recited in claim 11 wherein said first metal of said second component comprises lanthanum.

17. A composition as recited in claim 11 wherein said second metal of said second component comprises iron.

18. A composition as recited in claim 11 wherein said third metal of said second component is selected from the group consisting of titanium, vanadium, chromium, zirconium, molybdenum, and tungsten.

19. A composition as recited in claim 11 wherein said third metal of said second component is selected from the group consisting of titanium, vanadium, chromium and zirconium.

20. A composition as recited in claim 11 wherein said first component is present in said composition in an amount in the range of from about 10 to about 30 weight %, based on the total weight of said composition.

21. A composition as recited in claim 11 wherein said first component is present in said composition in an amount in the range of from about 15 to about 25 weight %, based on the total weight of said composition.

22. A composition as recited in claim 11 wherein said first component is present in said composition in an amount in the range of from about 18 to about 22 weight %, based on the total weight of said composition.

23. A method of preparing a composition comprising:
a) mixing:
(1) a first compound comprising a Group IIIB metal,
(2) a second compound comprising a Group VIII metal, and
(3) a third compound comprising a metal selected from the group consisting of a Group IVB metal, a Group VB metal and
a Group VIB metal, to thereby form a mixture thereof;
b) calcining said mixture to thereby form a calcined material ; and
c) incorporating a Group VIII metal onto said calcined material to thereby form said composition.

24. A method as recited in claim 23 wherein said Group VIII metal of step c) comprises a metal selected from the group consisting of iron, cobalt, ruthenium, and nickel.

25. A method as recited in claim 23 wherein said Group VIII metal of step c) comprises iron.

26. A method as recited in claim 23 wherein said Group IIIB metal of said first compound comprises lanthanum.

27. A method as recited in claim 23 wherein said Group VIII metal of said second compound comprises iron.

28. A method as recited in claim 23 wherein said metal of said third compound is selected from the group consisting of titanium, vanadium, chromium, zirconium, molybdenum, and tungsten.

29. A method as recited in claim 23 wherein said metal of said third compound is selected from the group consisting of titanium, vanadium, chromium and zirconium.

30. A method as recited in claim 23 wherein said Group VIII metal, incorporated onto said calcined material in step c), is present in said composition in an amount in the range of from about 10 to about 30 weight %, based on the total weight of said composition.

31. A composition prepared by the method of claim 23.

32. A process comprising contacting synthesis gas with the composition of claim 1, under reaction conditions, to thereby convert at least a portion of said synthesis gas to a hydrocarbon.

33. A process in accordance with claim 32 wherein said reaction conditions include a temperature in the range of from about 260° C. to about 380° C.

34. A process in accordance with claim 32 wherein said reaction conditions include a temperature in the range of from about 280° C. to about 360° C.

35. A process in accordance with claim 32 wherein said reaction conditions include a temperature in the range of from about 300° C. to about 340° C.

36. A process in accordance with claim 32 wherein said hydrocarbon contains in the range of from 2 to 4 carbon atoms per molecule.

37. A process comprising contacting synthesis gas with the composition of claim 31, under reaction conditions, to thereby convert at least a portion of said synthesis gas to a hydrocarbon.

38. A process in accordance with claim 37 wherein said reaction conditions include a temperature in the range of from about 260° C. to about 380° C.

39. A process in accordance with claim 37 wherein said reaction conditions include a temperature in the range of from about 280° C. to about 360° C.

40. A process in accordance with claim 37 wherein said reaction conditions include a temperature in the range of from about 300° C. to about 340° C.

41. A process in accordance with claim 37 wherein said hydrocarbon contains in the range of from 2 to 4 carbon atoms per molecule.
